(19) United States
(12) Patent Application Publication
(10) Pub. No.: US 2012/0022463 A1
(43) Pub. Date: Jan. 26, 2012

(54) SAFETY SHEATH FOR HYPODERMIC SYRINGE NEEDLE

(76) Inventor: Kuo Cheng WU, Kaohsiung City (TW)

(21) Appl. No.: 13/159,555

(22) Filed: Jun. 14, 2011

(30) Foreign Application Priority Data
Jul. 26, 2010 (TW) 099124498

(51) Int. Cl. A61M 5/32

(52) U.S. Cl. 604/197

(57) ABSTRACT

The present invention provides a safety sheath for hypodermic syringe needle comprising a proximal mounting ring, a distal mounting ring and a shielding sheath. The shielding sheath includes two symmetrical slits, two symmetrical semicircular aprons and an inward annular fillet, which is bisected into two symmetrical curved segments with crescent thickness, namely marginal thickness is less than central thickness. During sleeving the shielding sheath over the distal mounting ring and proximal mounting ring, both symmetrical semicircular aprons are outwardly hauled by outwards forces such that the bore profile of the inward annular fillet is dilated into circular shape, which will not only facilitate subsequent sleeving process without difficulty but also enhance the tightness for the latch of the shielding sheath with the distal mounting ring in locked manner without any possibility of mutually slipping displacement so that medical personnel in handling can prevent from accidental pricks by hypodermic needle.

### FIELD OF THE PRESENT INVENTION

The present invention relates to a safety sheath for hypodermic syringe needle, particularly for one to be used in a “jacketed safety hypodermic injection apparatus”. It provides a convenient mechanism for easily being introduced into automatic mass production without replacement of the existing machinery or equipments. Therefore, the intention of investing or transforming into production of “safety hypodermic injection apparatus” for the manufacturers of the conventional injection apparatus will be encouraged and spurred. Thus, medical personnel and medical waste handling personnel can be early freed from the menace of being possibly pricked by the hypodermic needle accidentally.

### BACKGROUND OF THE INVENTION

For medical personnel, having been inadvertently pricked by the hypodermic needle in handling hypodermic syringe becomes an unavoidable fact. According to report from USA Center for Disease Control and Prevention (CDC) that annual consumption of hypodermic syringe in USA is 6 billion pieces and 0.8 to 1.0 million accidental jabs with 18 thousand cases of hepatitis-B contagion. Accordingly, various types of “jacketed safety hypodermic injection apparatus” for preventing medical personnel from accidental jabs such as US patents in numbers of U.S. Pat. No. 2,571,653, U.S. Pat. No. 4,790,827, U.S. Pat. No. 4,631,057, U.S. Pat. No. 4,850,994, U.S. Pat. No. 4,915,702, U.S. Pat. No. 4,976,702, U.S. Pat. No. 4,998,920, U.S. Pat. No. 4,998,924, U.S. Pat. No. 5,207,646, U.S. Pat. No. 5,674,203, U.S. Pat. No. 5,735,823, U.S. Pat. No. 5,947,933, U.S. Pat. No. 6,149,630 and U.S. Pat. No. 6,702,786 have mushroomed.

However, some drawbacks in the foregoing various types of “jacketed safety hypodermic injection apparatus” cause related manufacturers reluctant to market. The vital drawbacks are as below:

Firstly, take one shield device of corresponding to referential number 10A in original specification in associated with FIGS. 4 through 6 of U.S. Pat. No. 4,976,702 as a typical conventional example of prior arts. The specification of U.S. Pat. No. 4,976,702 hereinafter is refereed to cited original specification. Please refer to FIGS. 1 through 3. The basic structure of the shield device comprises a translucent sheath 2 (corresponding to 56 in cited original specification) and a modified conventional hypodermic injection apparatus, which includes a hypodermic syringe 1 (corresponding to 12A in cited original specification), a hypodermic needle 1c (corresponding to 14 in cited original specification) and a piston or plunger P (corresponding to 30 in cited original specification), wherein: Said hypodermic syringe 1 is adapted to include a distal circumferential groove 1a (corresponding to 52 in cited original specification) and a proximal circumferential groove 1b (corresponding to 54 in cited original specification); and Said translucent sheath 2 includes a pair of flanges or lugs 2c (corresponding to 58 in cited original specification) and a plurality of protuberances or nipples 2b (corresponding to 62 in cited original specification) formed in the inner wall of a coupling cuff or sleeve rim 2a (as shown in FIG. 3). In practical operation, when the integral injection apparatus is prepared in ready to use, the translucent sheath 2 circumjacently sleeves the hypodermic syringe 1 in a retracted position such that the protuberances 2b in the coupling cuff 2a thereof latch with the proximal circumferential groove 1b of the hypodermic syringe 1 in immovably retained manner to avoid mutually slipping displacement due to inadvertently mishandling (as shown in FIG. 2). At this moment, the hypodermic needle 1c is exposed out of shield from the translucent sheath 2. When the integral injection apparatus finishes the hypodermic injection, the translucent sheath 2 can be released and forwardly slid over the hypodermic syringe 1 towards the hypodermic needle 1c to a extended position by hand such that the protuberances 2b in the coupling cuff 2a thereof latch with the distal circumferential groove 1a of the hypodermic syringe 1 in locked immobility without any possibility of mutually slipping displacement (as shown in FIG. 1). At this moment, the hypodermic needle 1c is entirely shielded by the translucent sheath 2 so that the medical personnel, who handles the hypodermic injection apparatus, can prevent from accidental jabs or pricks by the hypodermic needle 1c.

There are two drawbacks in such kind of structure as below:

1. Engineering Drawback:

Because the modified conventional hypodermic injection apparatus, whose hypodermic syringe 1 is adapted to include a distal circumferential groove 1a and a proximal circumferential groove 1b, an extra cost will be incurred for retrofitting or remolding conventional injecting apparatus as original manufacturing molds will be obsolete. According to regulations of the USA Food and Drag Administration (FDA), any modified product of the hypodermic injection apparatus is obliged to apply for reexamination with filing fee. For manufacturers of hypodermic injection apparatus, with uncertainty in waiting for statutorily stipulated processing period by the FDA, the intention for adjustment in manufacturing facility and process in comply with the retrofitting or remolding will be deterred to exhausted manner.

2. Assembly Drawback:

Please refer to FIGS. 4 and 5, wherein d1 denotes the inner diameter of the inner circumferential border formed by the protuberances 2b in the coupling cuff 2a of the translucent sheath 2 while d2 denotes the outer diameter of the hypodermic syringe 1. The fact is that d1 is less than d2.

During assembling process for circumjacently sleeving the translucent sheath 2 over the hypodermic syringe 1, with d1 being less than d2, both of the pair flanges 2c of the coupling cuff 2a must be firmly clipped by suitably pliers or tools to expand the coupling cuff 2a by outwardly radial forces simultaneously (as two arrowheads indicated Fv shown in FIG. 5) so that the profile of the coupling cuff 2a is deformed from an original round bore 2a into an oval bore 2a′ (as hypothetic line shown in FIG. 5).

At this moment, the translucent sheath 2 still can not circumjacently sleeve over the hypodermic syringe 1 because deformed oval bore 2a′ thereof is blocked by the front peripheral of the hypodermic syringe 1. Therefore, an axial force F towards the plunger P of the hypodermic syringe 1 must apply on the translucent sheath 2 to forcibly pass the protuberances 2b of the translucent sheath 2 over front peripheral of the hypodermic syringe 1 up to the extended position (as hypothetic line shown in FIG. 4), where the protuberances 2b in the coupling cuff 2a latch with the distal circumferential groove 1a of the hypodermic syringe 1 in locked manner (as shown in FIG. 1).

Even at this moment, the translucent sheath 2 still can not properly sleeve over the hypodermic syringe 1 in final position because the protuberances 2b in the coupling cuff 2a of the translucent sheath 2 is latched by the distal circumferential groove 1a of the hypodermic syringe 1. Therefore, another axial force F towards the plunger P of the hypodermic syringe 1 must also apply on the translucent sheath 2 to forcibly pass it over the extended position up to the retracted position, where the protuberances 2b in the coupling cuff 2a latch with the proximal circumferential groove 1b of the hypodermic syringe 1 in immovably retained manner (as shown in FIG. 2).

According to the statistics of the World Health Organization (WHO), global annual consumption quantity of the convention hypodermic injection apparatus is about 40 to 60 billion pieces. From foregoing description, such complicated difficulty in assembling process of the translucent sheath 2 and hypodermic syringe 1 is even hardly applied to current mass production of the “safety hypodermic injection apparatus”, not to mention that it can meet the requirement of the global annual production quantity.

Secondly, take shielded needle of U.S. Pat. No. 4,631,057 as another typical conventional example of prior arts. The specification of U.S. Pat. No. 4,631,057 hereinafter is refereed to cited original specification. As stated in the cited original specification that by adding only a collar and the needle guard without modification to the body of the injecting apparatus, a conventional injection apparatus can be provided with needle shielding features, please refer to FIGS. 6 and 7, other than a conventional hypodermic injection apparatus, which includes a hypodermic syringe 5 (corresponding to 11 in cited original specification), a hypodermic needle 5c (corresponding to 15 in cited original specification) and a piston or plunger P (corresponding to 17 in cited original specification), the basic structure of the shield device comprises a collar 3 (corresponding to 31 in cited original specification) and a needle guard 4 (corresponding to 33 in cited original specification), wherein: Said collar 3, which securely sleeves over the distal end peripheral of the hypodermic syringe 5, includes a distal annular groove 3a (corresponding to 35 in cited original specification) and a proximal annular groove 3b (corresponding to combinational 37 of 39 and 40 in cited original specification) such that the distal annular groove 3a is disposed over the peripheral thereof near the hypodermic needle 5c while the proximal annular groove 3b is disposed over the peripheral thereof near the plunger P; and Said needle guard 4, which snugly sleeves over peripheral of the hypodermic syringe 5 with the collar 3 in manner of being able to slide axially, includes a distal annular shoulder 4a (corresponding to 43 in cited original specification) and a proximal annular rib or projection 4b (corresponding to combination of 53 and 55 in cited original specification) such that the distal annular shoulder 4a is disposed in the inner wall thereof near the hypodermic needle 5c while proximal annular rib 4b is disposed in the inner wall thereof near the plunger P. In practical operation, when the integral injection apparatus is prepared in ready to use, the needle guard 4 circumjacently sleeves the collar 3 in a retracted position such that the distal annular shoulder 4a of the needle guard 4 latches with the distal annular groove 3a of the collar 3 in immovably retained manner to avoid mutually slipping displacement due to inadvertently mishandling (as shown in FIG. 6). At this moment, the hypodermic needle 5c is exposed out of shield from the needle guard 4 for easily handling injection. When the integral injection apparatus finishes the hypodermic injection, the needle guard 4 can be released and forwardly slid over the collar 3 towards the hypodermic needle 5c to a extended position by hand such that the proximal annular rib 4b of the needle guard 4 latches with the proximal annular groove 3b of the collar 3 in locked immobility without any possibility of mutually slipping displacement (as shown in FIG. 7). At this moment, the hypodermic needle 5c is entirely shielded by the needle guard 4 so that the medical personnel, who handles the hypodermic injection apparatus, can prevent from accidental jabs or pricks by the hypodermic needle 5c. Because no modification to the body of the injecting apparatus in this case, no extra cost will be incurred for retrofitting or remolding conventional injecting apparatus. However, there is serious assembly drawback in such kind of structure as below. Please refer to FIGS. 8 through 12, wherein d3 denotes the inner diameter of the proximal annular rib 4b of the needle guard 4 while d4 denotes the maximal outer diameter of the collar 3. The fact is that d3 is less than d4 (as shown in FIG. 9). During assembling process for circumjacently sleeving the needle guard 4 over the collar 3, with d3 being less than d4, two symmetrical sides in diametrically opposed fashion of the coupling cuff 4c must be firmly clipped by suitably pliers or tools to expand the coupling cuff 4c by outwardly radial forces simultaneously (as two arrowheads indicated Fv shown in FIG. 8) so that the profile of the coupling cuff 4c is deformed from an original round bore 4c into an oval bore 4c′ (as hypothetic line shown in FIG. 8). At this moment, the needle guard 4 still can not circumjacently sleeve over the collar 3 because deformed oval bore 4c′ thereof is blocked by the front peripheral of the collar 3 (as shown in FIG. 10). Therefore, an axial force F towards the plunger P of the hypodermic syringe 5 must apply on the needle guard 4 to forcibly pass the deformed oval bore 4c′ thereof over front peripheral of the collar 3 up to the extended position (as shown in FIG. 11), where the proximal annular rib 4b of the needle guard 4 latches with the proximal annular groove 3b of the collar 3 in locked manner (as shown in FIG. 7). Even at this moment, the needle guard 4 still can not properly sleeve over the collar 3 in final position because the proximal annular rib 4b of the needle guard 4 is latched by the proximal annular groove 3b of the collar 3 (as shown in FIG. 12). Therefore, another axial force F towards the plunger P of the hypodermic syringe 5 must also apply on the needle guard 4 to forcibly pass it over the extended position up to the retracted position, where the distal annular shoulder 4a of the needle guard 4 latches with the distal annular groove 3a of the collar 3 in immovably retained manner (as shown in FIG. 6). From foregoing description, the assembling process of the needle guard 4 and collar 3 on the hypodermic syringe 5 is indeed very complicated and difficult. Therefore, the shielded needle of U.S. Pat. No. 4,631,057 is not suitably applied in current mass production of the “safety hypodermic injection apparatus” either.

In conclusion the drawback description for two US patents as typical conventional injection apparatus of prior arts heretofore, the substantial factor in hindering the “jacketed safety hypodermic injection apparatus” into mass production and marketing promotion is bad structural design. Other than such bad structural design, current laws do not have compulsory regulations to require that manufacturer of the “safety hypodermic injection apparatus” is obligatory to retrofit existing conventional injection apparatus with drawback(s) mentioned above. Consequently, most manufacturers of the conventional injection apparatus are unwilling to invest or transform into production of “safety hypodermic injection apparatus”. Therefore, medical personnel and medical waste handling personnel are inevitable to continually stay under the menace of being possibly pricked by the hypodermic needle accidentally. With fully realizing these facts and addressing foregoing drawbacks, the applicant of the present invention zealously undertakes profound research and study via multiple tests of existing automatically proprietary machinery. Eventually, the present invention is successfully worked out.

### SUMMARY OF THE INVENTION

The primary object of the present invention is to provide a safety sheath for hypodermic syringe needle with basic structure comprising a proximal mounting ring, a distal mounting ring and a shielding sheath, wherein the proximal mounting ring, which sleeving over the bottom peripheral of the hypodermic syringe, has a proximal annular groove created on the circumferential surface; the distal mounting ring, which sleeving over the top peripheral of the hypodermic syringe, includes a conical frustum surface formed in the bottom section, an annular stopping curb formed in the top section and a distal annular groove created in the middle section thereof; and the shielding sheath, which sleeving over the peripheral walls of the proximal mounting ring and distal mounting ring, includes an inward annular fillet, a pair of symmetrical slits in diametrically opposed fashion, and a pair of symmetrical tensional recesses in diametrically opposed fashion, wherein the inward annular fillet is bisected into two symmetrical curved segments by the pair symmetrical slits such that the thickness of each curved segment is non-uniform like crescent; each of both tensional recesses is respectively located at symmetrical side in diametrically opposed fashion; and each slit is upwardly and longitudinally cut at the lower section of the circumferential wall; by means of the pair symmetrical slits, the lower section of the circumferential wall is bisected into two symmetrical semicircular aprons. During assembling process, both symmetrical semicircular aprons in the circumferential wall are outwardly hauled by two outwards forces such that the bore profile of the bottom face is dilated and deformed from original circular shape into oval shape, meanwhile the bore profile of the inward annular fillet is dilated into circular shape. Accordingly, both of the dilated oval bore profile of the bottom face and dilated circular bore profile of the inward annular fillet will not only facilitate subsequent sleeving process but also enhance the tightness for the latch of the shielding sheath with the distal mounting ring so that the medical personnel can prevent from accidental jabs or pricks by the hypodermic needle.

The other object of the present invention is to provide a safety sheath for hypodermic syringe needle with basic structure comprising a proximal mounting ring, a distal mounting ring and a shielding sheath as aforesaid, wherein each root of both bisected symmetrical semicircular aprons in the circumferential wall is terminated by each tensional recess, which not only absorbs the stress caused by outwards forces but also boosts recovering resilience for releasing deformation to ensure deformed bottom face of the shielding sheath is recovered to original shape without distortion.

### BRIEF DESCRIPTION OF THE DRAWINGS

FIG. 1 is the first sectional schematic view for the conventional structure of the U.S. Pat. No. 4,976,702.

FIG. 2 is the second sectional schematic view for the conventional structure of the U.S. Pat. No. 4,976,702.

FIG. 3 is a perspective schematic view partially showing a hypodermic syringe of the U.S. Pat. No. 4,976,702.

FIG. 4 is the first operational schematic view for the conventional U.S. Pat. No. 4,976,702.

FIG. 5 is the second operational schematic view for the conventional U.S. Pat. No. 4,976,702.

FIG. 6 is the first sectional schematic view for the conventional structure of the U.S. Pat. No. 4,631,057.

FIG. 7 is the second sectional schematic view for the conventional structure of the U.S. Pat. No. 4,631,057.

FIG. 8 is the first operational schematic view for the conventional U.S. Pat. No. 4,631,057.

FIG. 9 is the second operational schematic view for the conventional U.S. Pat. No. 4,631,057.

FIG. 10 is the third operational schematic view for the conventional U.S. Pat. No. 4,631,057.

FIG. 11 is the fourth operational schematic view for the conventional U.S. Pat. No. 4,631,057.

FIG. 12 is the fifth operational schematic view for the conventional U.S. Pat. No. 4,631,057.

FIG. 13 is an exploded perspective view of a safety sheath for hypodermic syringe needle according to a first preferred embodiment of the present invention.

FIG. 14 is a sectional view taken along line 14-14 as indicated in FIG. 13.

FIG. 15 is a sectional view taken along line 15-15 as indicated in FIG. 13.

FIG. 16 is a sectional view taken along line 16-16 as indicated in FIG. 13.

FIG. 17 is a sectional view taken along line 17-17 as indicated in FIG. 13.

FIG. 18 is a sectional view taken along line 18-18 as indicated in FIG. 17.

FIG. 19 is the first schematic view showing an assembly of the present invention in the above first preferred embodiment with a conventional hypodermic injection apparatus.

FIG. 20 is the second schematic view showing an assembly of the present invention in the above first preferred embodiment with a conventional hypodermic injection apparatus.

FIG. 21 is the third schematic view showing an assembly of the present invention in the above first preferred embodiment with a conventional hypodermic injection apparatus.

FIG. 22 is the fourth schematic view showing an assembly of the present invention in the above first preferred embodiment with a conventional hypodermic injection apparatus.

FIG. 23 is the fifth schematic view showing an assembly of the present invention in the above first preferred embodiment with a conventional hypodermic injection apparatus.

FIG. 24 is a sectional view taken along line 24-24 as indicated in FIG. 23.

FIG. 25 is the sixth schematic view showing an assembly of the present invention in the above first preferred embodiment with a conventional hypodermic injection apparatus.

FIG. 26 is the seventh schematic view showing an assembly of the present invention in the above first preferred embodiment with a conventional hypodermic injection apparatus.

FIG. 27 is the eighth schematic view showing an assembly of the present invention in the above first preferred embodiment with a conventional hypodermic injection apparatus.

FIG. 28 is the ninth schematic view showing an assembly of the present invention in the above first preferred embodiment with a conventional hypodermic injection apparatus.

FIG. 29 is the tenth schematic view showing an assembly of the present invention in the above first preferred embodiment with a conventional hypodermic injection apparatus.

FIG. 30 is a perspective schematic view showing completed assembly of the present invention in the above first preferred embodiment with a conventional hypodermic injection apparatus.

FIG. 31 is the first operational schematic view for the above first preferred embodiment of the present invention in practical usage.

FIG. 32 is the second operational schematic view for the above first preferred embodiment of the present invention in practical usage.

FIG. 33 is a sectional schematic view showing a modified shielding sheath for the second preferred embodiment of the present invention in practical usage.

FIG. 34 is an operational schematic view for the above second preferred embodiment of the present invention in practical usage.

FIG. 35 is a sectional schematic view showing an adapted shielding sheath for the third preferred embodiment of the present invention in practical usage.

FIG. 36 is an operational schematic view for the above third preferred embodiment of the present invention in practical usage.

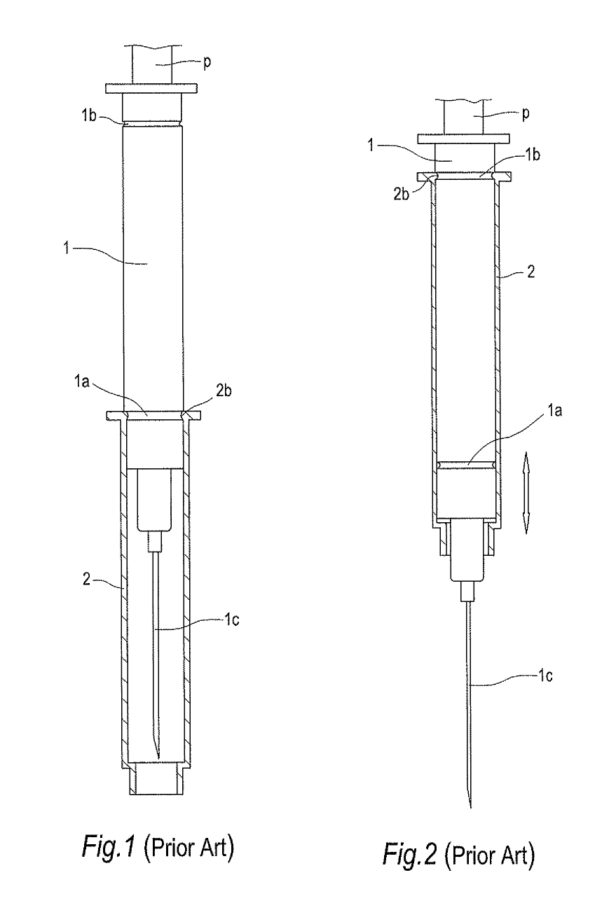

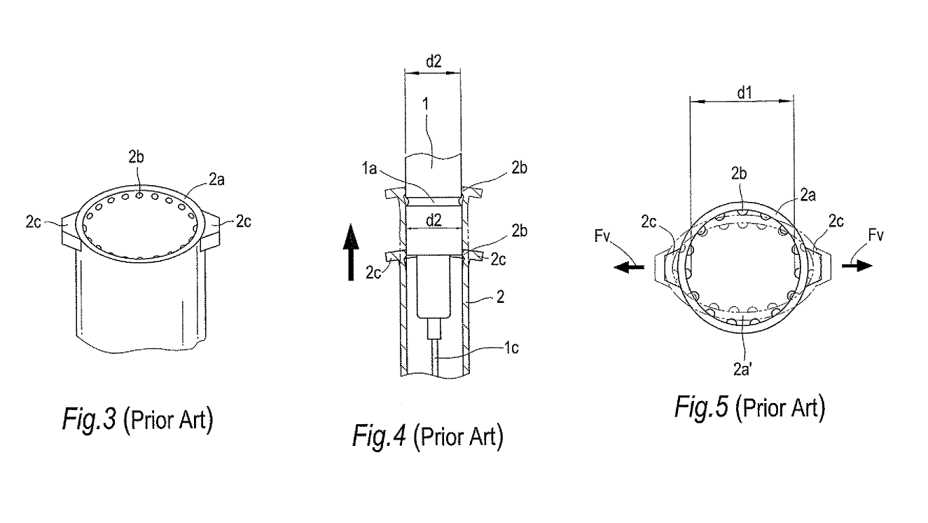

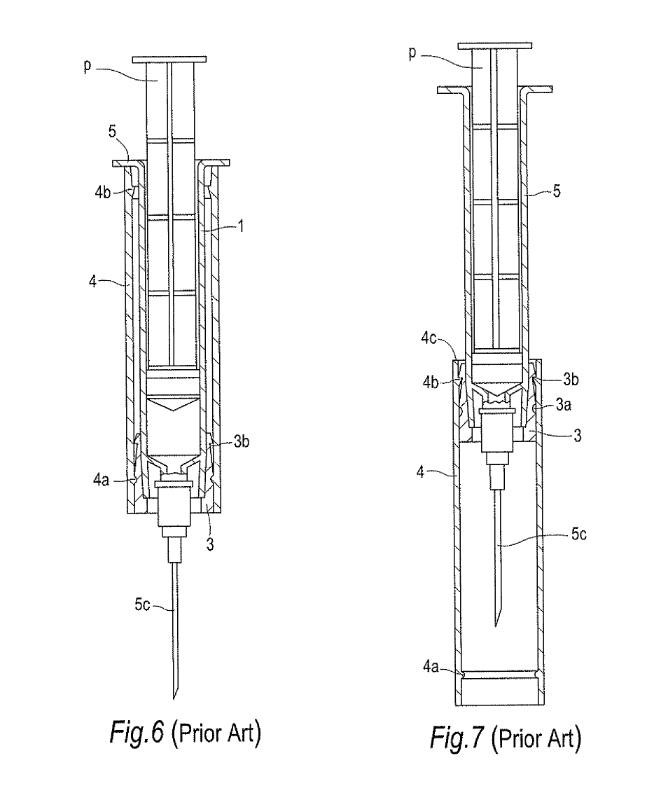

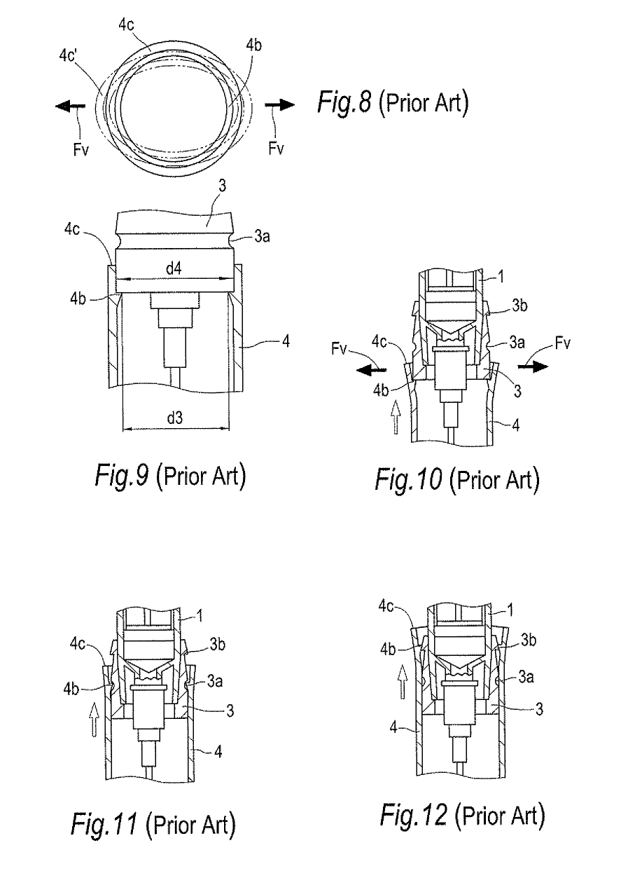

### DETAILED DESCRIPTION OF THE PREFERRED EMBODIMENTS

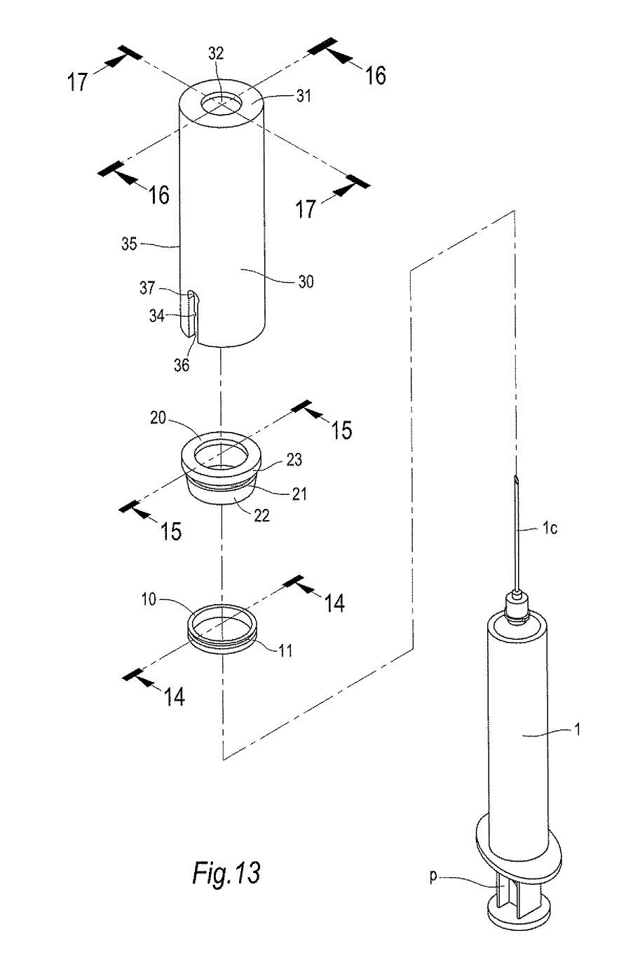

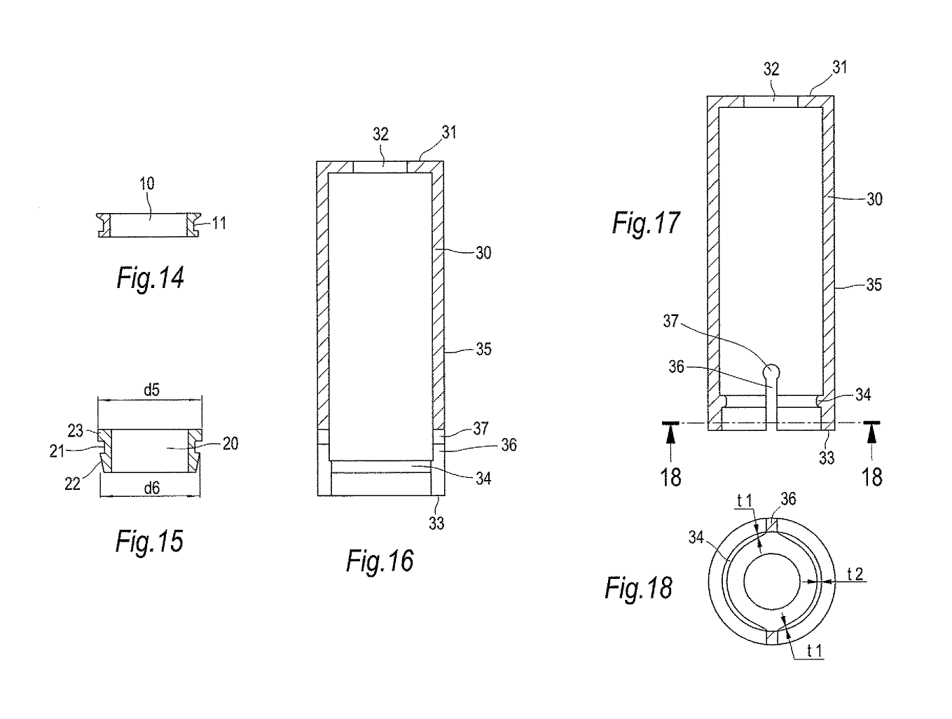

Please refer to FIGS. 13 through 18. Other than a conventional hypodermic injection apparatus, which includes a hypodermic syringe 1 with a pair of finger resting wings, a hypodermic needle 1c with a needle cap and a piston or plunger P with a thumb rest, the basic structure of a safety sheath for hypodermic syringe needle according to a first preferred embodiment of the present invention comprises a proximal mounting ring 10, a distal mounting ring 20 and a shielding sheath 30.

Referring to FIGS. 13 to 18, the proximal mounting ring 10 is a transparent or translucent hollow circular element with inner diameter equaling to outer diameter of the hypodermic syringe 1 so that it circumjacently sleeves over the bottom peripheral of the hypodermic syringe 1 in tight manner, has a proximal annular groove 11 created on the circumferential surface;

The distal mounting ring 20 is a transparent or translucent hollow circular element with inner diameter equaling to outer diameter of the hypodermic syringe 1 so that it circumjacently sleeves over the top peripheral of the hypodermic syringe 1 in tight manner, includes a downwardly tapered conical frustum surface 22 formed in the bottom section, an annular stopping curb 23 formed in the top section and a distal annular groove 21 created in the middle section thereof to sandwich between the annular stopping curb 23 and conical frustum surface 22 in abutting manner such that the outer diameter d5 of the annular stopping curb 23 is bigger than the maximal outer diameter d6 of the conical frustum surface 22 (as shown in FIG. 15); and

The shielding sheath 30 is a hollow cylinder encompassed by a top face 31 with a central perforation 32, a bottom face 33 and a circumferential wall 35 in circumjacently sleeving over the peripheral walls of the proximal mounting ring 10 and distal mounting ring 20 such that the overall length thereof is shorter than the overall length of the hypodermic syringe 1, includes an inward annular fillet 34, a pair of symmetrical slits 36 in diametrically opposed fashion, and a pair of symmetrical tensional recesses 37 in diametrically opposed fashion, wherein the inward annular fillet 34, which projects from the inner wall of the shielding sheath 30 near the bottom face 33 (as shown in FIG. 16), is bisected into two symmetrical curved segments by the pair symmetrical slits 36 such that the thickness of each curved segment is non-uniform like crescent, namely marginal thickness t1 is less than central thickness t2 (as shown in FIG. 18); each tensional recess 37 is created at the circumferential wall 35 in suitable height position from the bottom face 33 so that each of both tensional recesses 37 is respectively located at symmetrical side in diametrically opposed fashion (as shown in FIG. 17); and each slit 36 is upwardly and longitudinally cut at the lower section of the circumferential wall 35 from the bottom face 33 up to the bottom edge of the tensional recesses 37 such that the width thereof is less than bore diameter of the tensional recesses 37 (as shown in FIGS. 17 and 18); by means of the pair symmetrical slits 36, the lower section of the circumferential wall 35 is bisected into two symmetrical semicircular aprons.

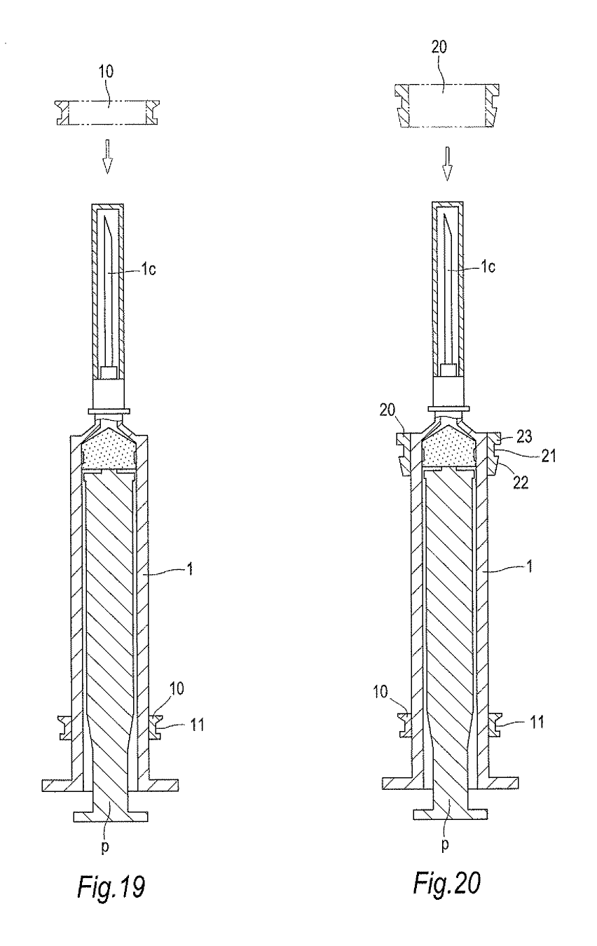

Please refer to FIGS. 19 through 25. The assembling process for the present invention in the above first exemplary preferred embodiment with a conventional hypodermic injection apparatus is described in stepwise as below:

Step a:

Circumjacently sleeve the proximal mounting ring 10 over the bottom peripheral, where near the thumb rest of the plunger P, of the hypodermic syringe 1 in tight manner (as shown in FIG. 19);

Step b:

Circumjacently sleeve the distal mounting ring 20 over the top peripheral, of the hypodermic syringe 1 in tight manner (as shown in FIG. 20);

Step c:

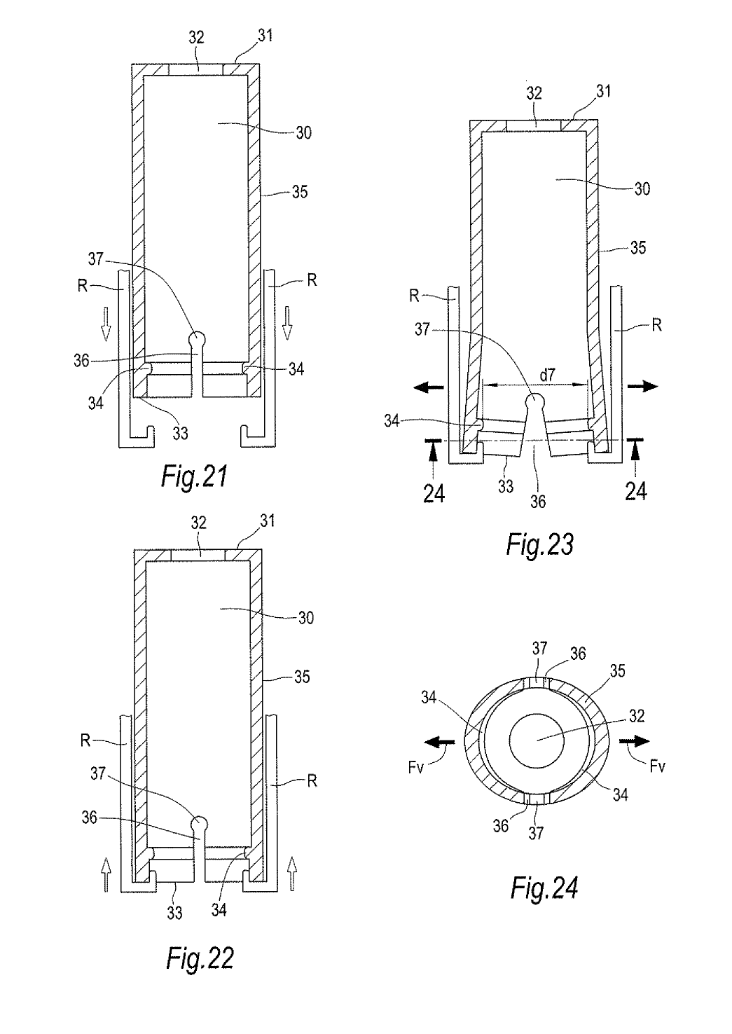

Employ a pair of symmetrical hooked robot arms or manipulators R to clip and hold lower inner ends of two symmetrical semicircular aprons in the circumferential wall 35 (as shown in FIGS. 21 and 22), then outwardly haul two symmetrical semicircular aprons in the circumferential wall 35 by two outwards forces Fv to simultaneously dilate the bottom face 33 of the shielding sheath 30 and the inner diameter d7 of the inward annular fillet 34 in deformed manner (as shown in FIG. 23); At this moment, the bore profile of the bottom face 33 is deformed from original circular shape into oval shape, meanwhile the bore profile of the inward annular fillet 34 is dilated into circular shape (as shown in FIG. 24) because the thickness of each bisected symmetrical curved segments of the inward annular fillet 34 is non-uniform like crescent, namely marginal thickness t1 is less than central thickness t2; Accordingly, both of the dilated oval bore profile of the bottom face 33 and dilated circular bore profile of the inward annular fillet 34 will facilitate subsequent sleeving process instead of being hindered by the distal mounting ring 20 and proximal mounting ring 10; Besides, each root of both bisected symmetrical semicircular aprons in the circumferential wall 35 is terminated by each tensional recess 37, which not only absorbs the stress caused by outwards forces Fv but also boosts recovering resilience for releasing deformation to ensure deformed bottom face 33 of the shielding sheath 30 is recovered to original shape without distortion;

Step d:

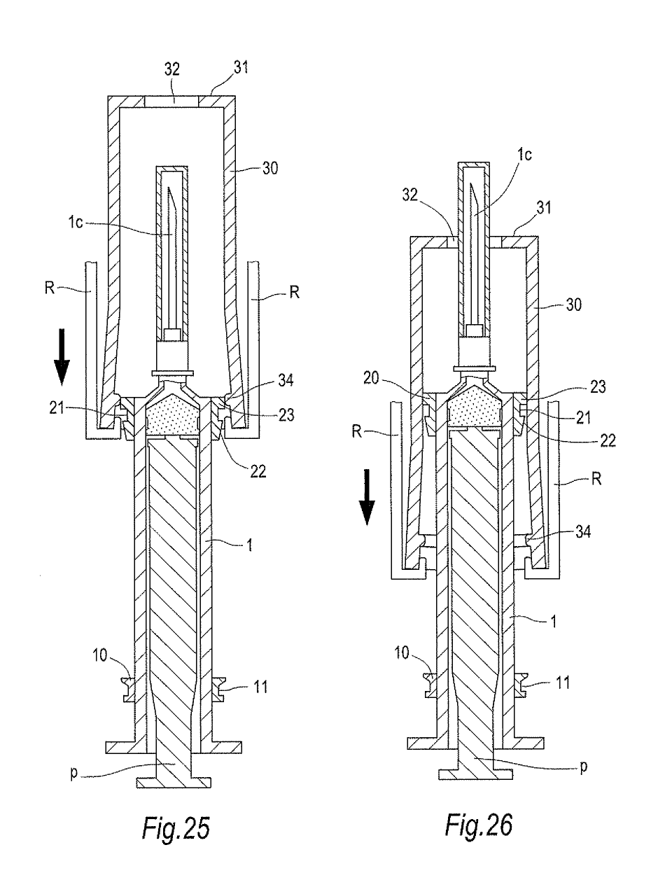

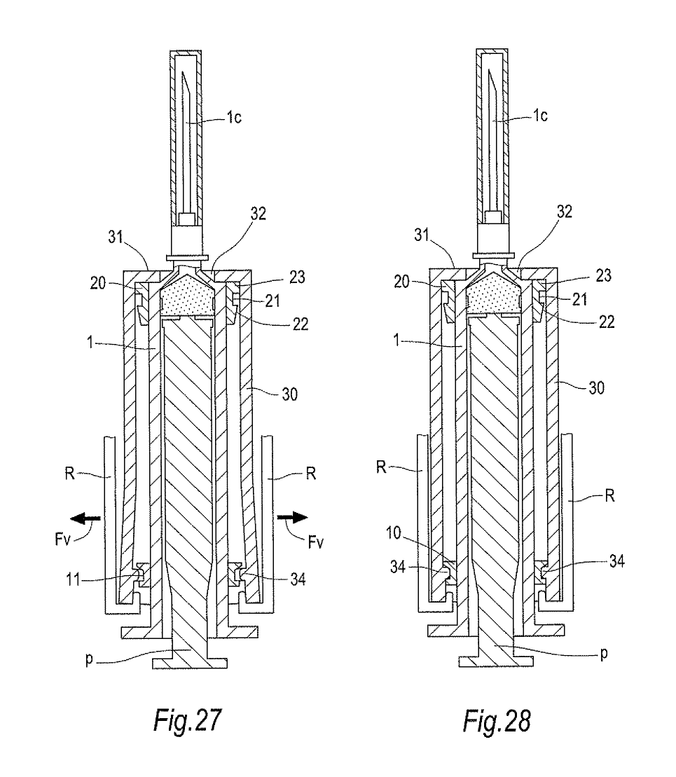

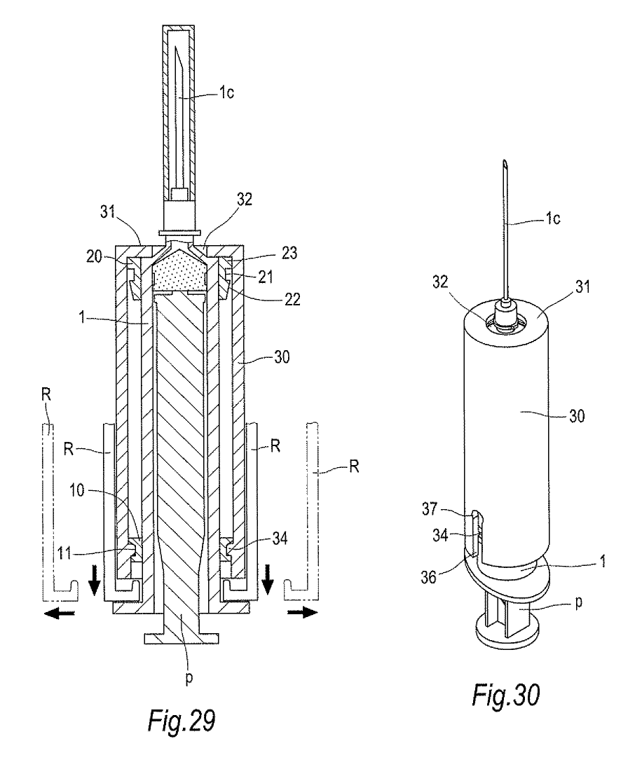

Downwardly align and move the shielding sheath 30 by the pair of symmetrical hooked robot arms or manipulators R to pass the peripheral of the distal mounting ring 20 in circumjacently sleeving manner (as shown in FIG. 25); downwardly move the shielding sheath 30 continually up to pass the peripheral of the proximal mounting ring 10 in circumjacently sleeving manner (as shown in FIGS. 26 and 27); release the outwards forces Fv applied by the pair of symmetrical hooked robot arms or manipulators R to recover the deformed bottom face 33 of the shielding sheath 30 to original shape, and meanwhile to latch the inward annular fillet 34 into the proximal annular groove 11 of the proximal mounting ring 10 (as shown in FIG. 28); downwardly detach away and outwardly move away out the pair of symmetrical hooked robot arms or manipulators R from the shielding sheath 30 (as indicated by arrowheads and hypothetic silhouette shown in FIG. 29); and finally the assembling process for the present invention in the first exemplary preferred embodiment with a conventional hypodermic injection apparatus is completed (as shown in FIG. 30).

Wherein, the steps a and b, the fit between the shielding sheath 30 and distal mounting ring 20 as well as the fit between the shielding sheath 30 and proximal mounting ring 10 are in “transition fit” or “interference fit” because the inner diameters of the distal mounting ring 20 and proximal mounting ring 10 are equals the inner diameter of the shielding sheath 30. Optionally, the fit strength between the shielding sheath 30 and distal mounting ring 20 as well as the fit strength between the shielding sheath 30 and proximal mounting ring 10 can be enhanced by any suitable viscous glue. Moreover, any suitable existing automatic proprietary machinery or equipments can be introduced into all processing steps a through d to increase efficiency and productivity. Therefore, the present invention is substantially applicable to mass production of the “safety hypodermic injection apparatus”.

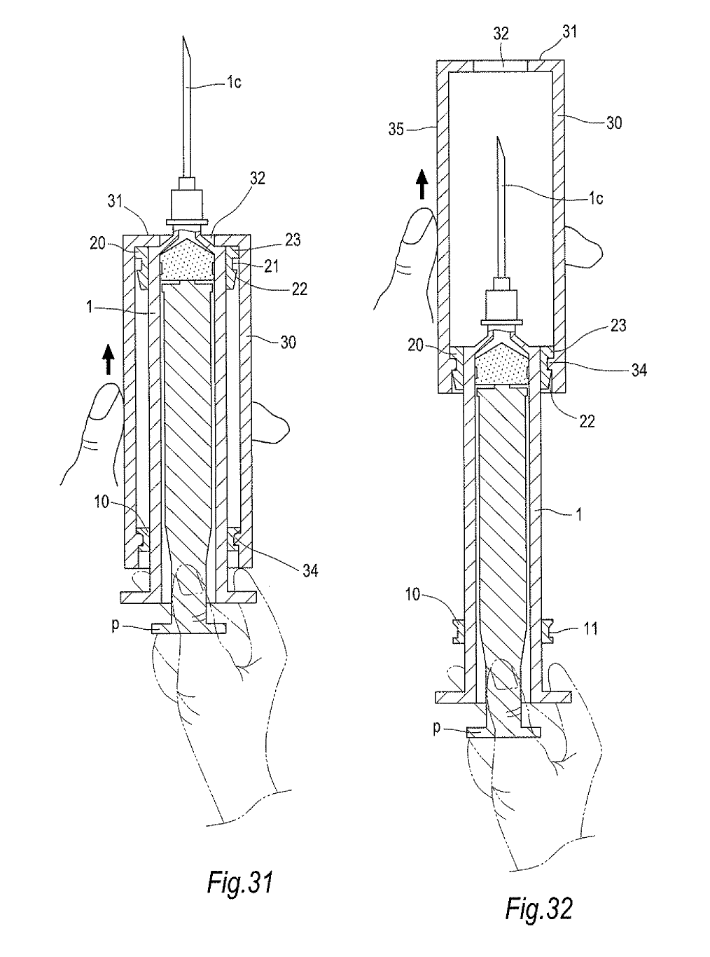

Please refer to FIGS. 31 and 32. The operational mode for the present invention in the above first preferred embodiment with a conventional hypodermic injection apparatus is described as below. When the integral injection apparatus is prepared in ready to use, the shielding sheath 30 circumjacently sleeves the distal mounting ring 20 and proximal mounting ring 10 in a retracted position such that the hypodermic needle 1c is exposed out of shield from the shielding sheath 30 for easily handling injection (as shown in FIG. 31). When the integral injection apparatus finishes the hypodermic injection, the shielding sheath 30 can be released and forwardly slid over the hypodermic syringe 1 towards the hypodermic needle 1c to a extended position by hands such that the inward annular fillet 34 of the shielding sheath 30 latches with the distal annular groove 21 of the distal mounting ring 20 in locked immobility without any possibility of mutually slipping displacement (as shown in FIG. 32). In a typical exemplary operation, when the integral injection apparatus finishes the hypodermic injection, firstly hold the shielding sheath 30 by left hand and hold the hypodermic syringe 1 by right hand with index finger and middle finger hooking the pair finger resting wings (as shown in FIG. 31); and then backwardly pull the hypodermic syringe 1 along the shielding sheath 30 in longitudinal manner or forwardly push the shielding sheath 30 along the hypodermic syringe 1 in longitudinal manner until the extended position such that the inward annular fillet 34 of the shielding sheath 30 latches with the distal annular groove 21 of the distal mounting ring 20 in locked condition so that the hypodermic needle 1c is entirely shielded by the shielding sheath 30 (as shown in FIG. 32). Please refer back to the distal mounting ring 20 in the FIG. 15, which shows the outer diameter d6 of the downwardly tapered conical frustum surface 22 thereof is less than the outer diameter d5 of the annular stopping curb 23 thereof. With downwardly tapered design of the conical frustum surface 22, the circumjacently sleeving movement for the shielding sheath 30 over the conical frustum surface 22 of the distal mounting ring 20 is easily facilitated in smooth way. With the outer diameter d6 of the conical frustum surface 22 being less than the outer diameter d5 of the annular stopping curb 23, once the inward annular fillet 34 of the shielding sheath 30 is latched with the distal annular groove 21 of the distal mounting ring 20, it will be locked there in immobility manner without any possibility of mutually slipping displacement so that the medical personnel, who handles the hypodermic injection apparatus, can prevent from accidental jabs or pricks by the hypodermic needle 1c.

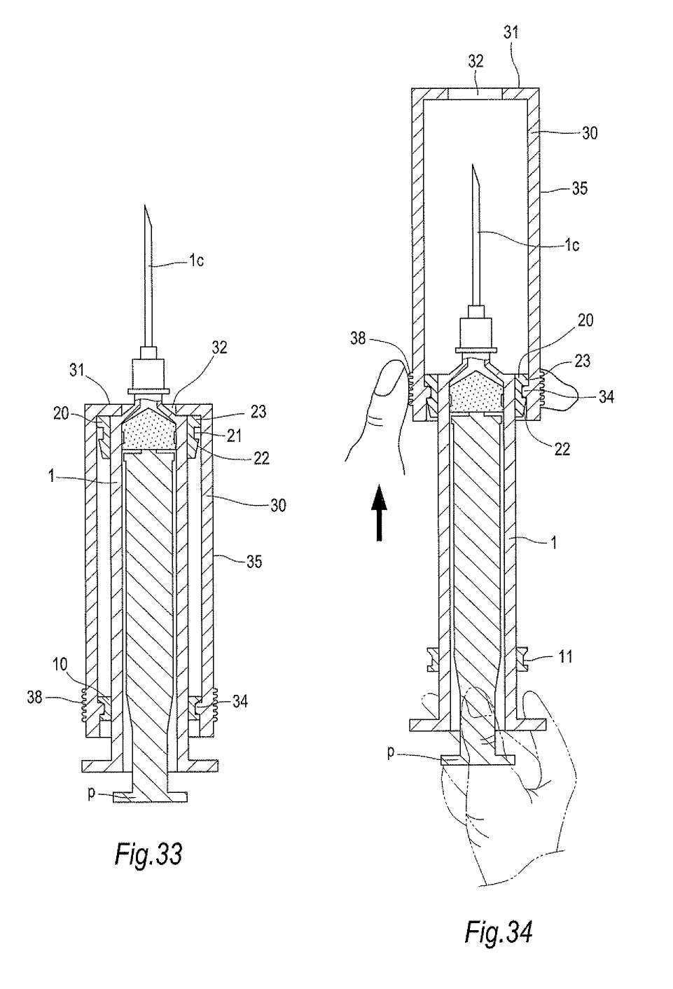

Please refer to FIG. 33, which shows a modified shielding sheath for the second preferred embodiment of the present invention in practical usage, wherein said circumferential wall 35 of the shielding sheath 30 is further modified with multiple corrugated circumferential collars 38. Thereby, the friction of the hand holding on the shielding sheath 30 can be increased so that backwardly pulling movement for the hypodermic syringe 1 along the shielding sheath 30 in longitudinal manner or forwardly pushing movement for the shielding sheath 30 along the hypodermic syringe 1 in longitudinal manner can be facilitated (as shown in FIG. 34).

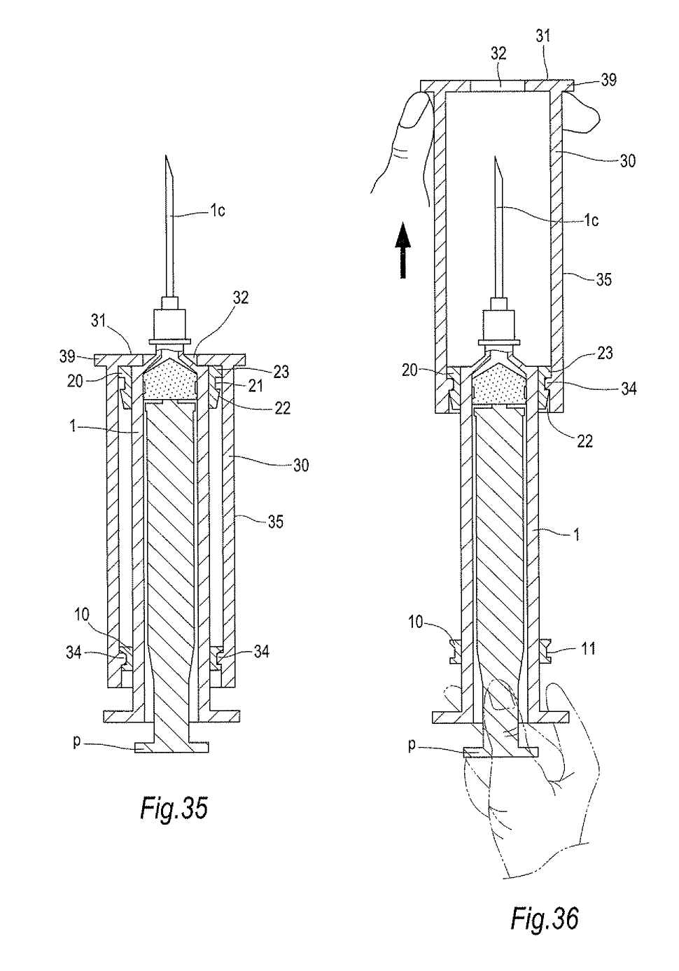

Please refer to FIGS. 35 and 36, which show an adapted shielding sheath for the third preferred embodiment of the present invention in practical usage, wherein near the top face 31 of said shielding sheath 30, the distal end for the circumferential wall 35 thereof is further adapted with an annular flanged shoulder 39. Thereby, the annular flanged shoulder 39 provides not only an additional curb for hand in holding the shielding sheath 30 but also a finger resting prop for easily pushing the shielding sheath 30 forwardly along the hypodermic syringe 1 in longitudinal manner (as shown in FIG. 36).

In conclusion the disclosure heretofore, the present invention not only resolving existing drawbacks in the conventional “jacketed safety hypodermic injection apparatus” but also provides a convenient mechanism for easily being introduced into automatic mass production without replacement of the existing machinery or equipments. Therefore, the intention of investing or transforming into production of “safety hypodermic injection apparatus” for the manufacturers of the conventional injection apparatus will be encouraged and spurred. Thus, the present invention indeed meets the basic patentable criterion. Accordingly, we here submit the patent application per related laws.

What is claimed is:

1. A safety sheath for hypodermic syringe needle, other than a conventional hypodermic injection apparatus, which includes a hypodermic syringe with a pair of finger resting wings, a hypodermic needle with a needle cap and a piston or plunger with a thumb rest, the basic structure comprising:
a proximal mounting ring;
a distal mounting ring; and
a shielding sheath,
wherein said proximal mounting ring is a hollow circular element circumjacently sleeving over the bottom peripheral of the hypodermic syringe in tight manner, has a proximal annular groove created on the circumferential surface,
wherein said distal mounting ring is a hollow circular element circumjacently sleeving over the top peripheral of the hypodermic syringe in tight manner, includes a downwardly tapered conical frustum surface formed in the bottom section, an annular stopping curb formed in the top section and a distal annular groove created in the middle section thereof to sandwich between the annular stopping curb and conical frustum surface in abutting manner such that the outer diameter of the annular stopping curb is bigger than the maximal outer diameter of the conical frustum surface, and
wherein said shielding sheath is a hollow cylinder encompassed by a top face with a central perforation, a bottom face and a circumferential wall in circumjacently sleeving over the peripheral walls of the proximal mounting ring and distal mounting ring such that the overall length thereof is shorter than the overall length of the hypodermic syringe, includes an inward annular fillet, a pair of symmetrical slits in diametrically opposed fashion, and a pair of symmetrical tensional recesses in diametrically opposed fashion, wherein the inward annular fillet, which projects from the inner wall of the shielding sheath near the bottom face, is bisected into two symmetrical curved segments by the pair symmetrical slits such that the thickness of each curved segment is non-uniform like crescent, namely marginal thickness is less than central thickness; each tensional recess is created at the circumferential wall in suitable height position from the bottom face so that each of both tensional recesses is respectively located at symmetrical side in diametrically opposed fashion; and each slit is upwardly and longitudinally cut at the lower section of the circumferential wall from the bottom face up to the bottom edge of the tensional recesses such that the width thereof is less than bore diameter of the tensional recesses; by means of the pair symmetrical slits, the lower section of the circumferential wall is bisected into two symmetrical semicircular aprons.

2. A safety sheath for hypodermic syringe needle as claimed in claim 1, said circumferential wall of said shielding sheath is further modified with multiple corrugated circumferential collars.

3. A safety sheath for hypodermic syringe needle as claimed in claim 1, said distal end for the circumferential wall of said shielding sheath is further adapted with an annular flanged shoulder.
